(19) United States
(12) Patent Application Publication
(10) Pub. No.: US 2010/0298463 A1
(43) Pub. Date: Nov. 25, 2010

(54) DENTURE ADHESIVE COMPOSITION

(75) Inventors: Prasad Adusumilli, Parsippany, NJ (US); Stanley J. Lech, Parsippany, NJ (US); Fang Deng, Parsippany, NJ (US)

Correspondence Address: GlaxoSmithKline;GLOBAL PATENTS -US, UW2220, P. O. BOX 1539, KING OF PRUSSIA, PA, 19406-0939, US

(73) Assignee: SmithKline Beecham Corporation

(21) Appl. No.: 12/375,250

(22) PCT Filed: Jul. 30, 2007
(86) PCT No.: PCT/US07/74699

§ 371 Date: Jan. 27, 2009

Related U.S. Application Data
(60) Provisional application No. 60/834,779, filed on Jul. 31, 2006.

(51) Int. Cl. A61K 6/08

(52) U.S. Cl. 523/120

(57) ABSTRACT

The present invention is directed to an improved denture adhesive composition. In particular, this invention is directed to a denture adhesive composition free of a hydrocarbon vehicle, such as petrolatum or mineral oil.

### FIELD OF THE INVENTION

The present invention is directed to an improved denture adhesive composition.

### BACKGROUND OF THE INVENTION

Dentures are substitutes for missing teeth and serve as replacement for all or some of the teeth found in the oral cavity. Over time, even well fitting dentures can become ill fitting due to natural shrinkage and changes in the gum or mucosal tissues. Therefore, adherent creams, liquids, powders, and “liners” are often used to secure dentures within the mouth. Liners are denture adhesives in the form of a thin film, strip, or wafer with a certain desirable strength and integrity for the liner to be placed in between the prosthesis and the palate or jaw, which swells in the mouth fluid and provides the adhesive effect.

Traditionally, dentures within the mouth were secured by using adherent powders prepared from natural gum materials such as karaya, acacia or tragacanth gum. These materials have the property of swelling to many times their original volume upon the addition of water to form a gelatinous or mucilaginous mass. Cream forms of the adherent, prepared from finely ground particles of the gums, were also available and used instead of the powder compositions.

Over the years, there have been numerous improvements over the above-described simple denture adhesive formulations. U.S. Pat. No. 2,978,812 discloses a denture fixative composition which includes an ethylene oxide polymer having a molecular weight between 50,000 and 5,000,000 in an amount preferably comprising at least 50% of the active fixative material.

GB Patent No. 1,444,485 discloses a fixing agent comprising a solution of 4 to 44 wt. % of a polyvinyl pyrrolidone (“PVP”). U.S. Pat. No. 3,003,988 describes the use of mixed salts of more than 40 wt. % of a water-insoluble water-sensitized polymeric material consisting essentially of lower alkyl vinyl ether maleic anhydride copolymers. U.S. Pat. No. 5,001,170 discloses a substantially anhydrous mixture of about 20-40 wt. % of methyl vinyl ether maleic acid copolymer, 20-40 wt. % of PVP, and 20-40 wt % of ethylene oxide polymer.

Recent improvements include the use of a lower alkyl vinyl ether maleic acid, anhydride, or salt polymer or mixtures thereof and one or more metallic salts selected from the group consisting of calcium, magnesium, strontium, sodium, potassium, zirconium, and zinc, or mixtures thereof. U.S. Pat. No. 5,073,604 discloses a denture adhesive composition with mixed partial salts of a lower alkyl vinyl ether maleic acid copolymer, wherein said partial salts contain as the cationic salt function, (a) from about 10% to about 65% zinc or strontium cations; and (b) from about 10% to about 75% calcium cations of the total initial carboxyl groups reacted.

Many efforts have been made over the years to develop denture adhesive compositions with improved adhesion or hold between the oral mucosa and denture, better sensate attributes and mouth feel, minimized oozing of the adhesive materials from under the dental plate during denture application, reduced messiness and difficulties of removing the residual adhesive materials from the mouth and dentures, better sealing of the food particles trapped between the denture and the oral cavity of the denture wearer.

The majority of currently commercial denture adhesive products are oil/wax based water-soluble polymer suspensions or cream pastes. Even though these products can provide satisfactory hold for dentures, they tend to spread out or ooze when an excess amount of the product has been applied to the dentures. In addition, once many of these products have been inserted into the mouth, they have a metallic or medicine-like taste and cause oily messiness in the mouth. Since the cream products are made from water-soluble polymer suspensions, they also have the grittiness caused by those polymer particles.

Furthermore, persistent dry mouth, commonly referred to as xerostomia, is a relatively common complaint that can make the wearing of dentures very uncomfortable for affected individuals. In order for dentures to be comfortably stable in the mouth, intimate contact between the dentures and the underlying gums must be achieved and maintained during chewing, swallowing, and speaking. The presence of an adequate amount and consistency of saliva between the dentures and gums is essential. In the absence of saliva's lubricating effects, the gum, cheek and lip tissues may become irritated as the dentures move during chewing, swallowing and speaking. The non-hydrocarbon based denture adhesives described herein can not only provide the necessary lubrication between denture and gum tissue, but also keep the muco-tissue hydrated and stimulate the saliva flow which is beneficial to the overall heath of mouth.

In view of the disadvantages of current denture adhesive formulations, it is desirable to develop new generations of denture adhesive materials, in particular, ones without hydrocarbon-based vehicles, such as, mineral oil and other wax-like ingredients as is currently used in cream formulas. This new generation of denture adhesives uses water, glycerin, propylene glycol or low molecular weight polyethylene glycol, or combinations thereof, as the polymer delivery system or suspension medium for the formula, and is not hydrocarbon-based. In that regard, the inventors have discovered that by selecting proper polymer mixtures, a denture adhesive effective clear or translucent gel and/or paste can be made using water, glycerin, propylene glycol, or a low molecular weight polyethylene glycol, alone or in combinations thereof. The inventors have found that these denture adhesives have better or equivalent adhesive properties and hold relative to currently marketed denture adhesive products having hydrocarbon-based vehicles. Advantageously, the denture adhesive composition disclosed herein has a homogeneous appearance as either a clear or transparent gel, is non-gritty, has elastomeric properties for a cushioning effect in the mouth, does not use a wax-based or mineral oil vehicle, and has a better cohesive strength of the adhesive network with less or no ooze. In addition, these hydrocarbon-free denture adhesives provide mouth moisturizing/lubricating properties, better mouth feel, improved sensate attributes and a better taste profile to denture wears than the current marketed products.

Applicants have found that the combination of polyethylene oxide (“PEO”) and hydroxypropylmethylcellulose (“HPMC”) polymers in a hydrocarbon-free denture adhesive vehicle, surprisingly yields a denture adhesive formulation which has advantageous properties, improved aesthetics and comparable adhesive strength to denture adhesive formulations using hydrocarbon-based vehicles.

### SUMMARY OF THE INVENTION

In one aspect, this invention relates to a denture adhesive composition free of hydrocarbon vehicles such as mineral oil or petrolatum.

In another aspect, this invention relates to a denture adhesive composition free of a hydrocarbon vehicle and having a combination of at least two polymers, one which has adhesive properties, the other which has cohesive properties.

In yet another aspect, this invention relates to a denture adhesive composition free of a hydrocarbon vehicle having a combination of polyethylene oxide and hydroxypropylmethyl cellulose.

In still another aspect, this invention relates to the a method for adhering a denture to the oral mucosa resulting from the use of the new composition of the present invention which is a denture adhesive composition free of hydrocarbon vehicles such as mineral oil or petrolatum.

In still yet another aspect, this invention relates to a method of making the denture adhesive composition as described herein.

### DETAILED DESCRIPTION OF THE INVENTION

The term “adhesive properties” is used herein to refer to the property of any substance, inorganic, organic, natural or synthetic, that is capable of bonding other substances together by surface attachment. In the instant situation, this refers to the ability of a denture adhesive product to hold together the surfaces of a denture and mucosal tissue.

The term “cohesive properties” is used herein to refer to the property of a material to stick together within itself. In our case, this refers to the ability of a denture adhesive product to maintain its integrity during its application.

While one of skill in the art would understand that both HPMC and Polyox have adhesive and cohesive features, in the context of this invention, HPMC is suitably the adhesive polymer component and Polyox is suitably the cohesive polymer component of one embodiment of this invention.

The term “dental appliance” is used herein to refer to dentures or partial dentures, artificial teeth, removable orthodontic bridges and denture plates, both upper and lower types, orthodontic retainers and appliances, protective mouthguards, nightguards to prevent bruxism and/or Temporomandibular joint (TMJ) disorder, and the like

The term “hydrophilic polymers” is used herein to refer to those polymers that have certain affinities to water molecules or those polymers that can attract water molecules.

The term “water-soluble polymers” is used herein to refer to those polymers that can be dissolved in water completely thereby forming a homogeneous mixture with water.

The terms “polyethylene oxide” and “ethylene oxide polymer” are used herein interchangeably.

The term “water-miscible solvent” is used herein to refer to those solvents or medium that can form a homogeneous mixture with water.

The present invention discloses novel denture adhesive compositions containing as the vehicle, water, glycerin, propylene glycol, or a low molecular weight polyethylene glycol, alone or in combination. Upon hydration by saliva or during actual use in the oral environment, the inventive material hydrates and becomes tacky and develops adhesiveness between the mucosal tissue and the denture.

Suitably the denture adhesive vehicle is water, glycerin, propylene glycol, or a low molecular weight polyethylene glycol, alone or in combination. In one embodiment of the invention, the denture adhesive vehicle comprises water alone as the vehicle. In one embodiment of the invention, the composition comprises between 20 and 80 wt. % of water alone as the vehicle. In another embodiment, the composition comprises water and one or more water-miscible solvents. The water-miscible solvent is suitably selected from glycerin, propylene glycol, low molecular weight polyethylene glycol, ethanol, Sorbitol and other polyhydroxy compounds. Suitably, the molecular weight for the polyethylene glycol solvent ranges between 200-800. In one embodiment of the invention, the composition comprises between 20 and 30 wt. % water and between 35 and 40 wt. % glycerin. In one embodiment of the invention, the composition comprises glycerin alone as a vehicle in an amount between 45 and 65 wt. % of the composition. In one embodiment of the invention, the composition comprises propylene glycol alone as a vehicle in an amount between 50 and 60 wt. % of the composition.

The hydrocarbon-free vehicle is suitably in combination with a hydrophilic or water-soluble polymer or polymer combination manufactured to form a paste or gel. The polymeric material used in the composition includes, but is not limited to, certain categories of polymers.

A first suitable polymer material is selected from water-soluble or water dispersible cellulose derivatives, such as hydroxypropyl methylcellulose (“HPMC”), carboxymethyl-cellulose (“CMC”), hydroxyethylcellulose (“HEC”), hydroxypropylcellulose (“HPC”), hydroxyethylmethylcellulose (“HEMC”), hydroxyethylmethylcellulose (“HEMC”), methyl-cellulose (“MC”), methylcarboxymethyl cellulose (“MCMC”), hydroxyethylcarboxymethyl cellulose (“HECMC”), hydroxyethylmethylcarboxy methylcellulose (“HEMCMC”), sulfoethylcarboxymethyl cellulose (“SECMC”), hydroxyethylhydroxypropyl cellulose (“HEHPC”), hydroxyethylethyl cellulose (“HEEC”), hydroxyethylsulfoethyl cellulose (“HESEC”), or combinations thereof. In one embodiment of the invention, the polymer material is HPMC.

A second suitable polymer material is selected from water-soluble or water dispersible polyethylene oxide (“PEO”) homopolymers or copolymers, such as polyethylene glycol and its derivatives, PolyOx® polymers, water-soluble or water dispersible polypropylene oxide homopolymers or copolymers such as Poloxamer®, and Pluronic® polymers. In one embodiment of the invention, the polymer is PEO.

A third suitable polymer material is selected from water-soluble or water dispersible poly(methylvinyl ether-co-maleic acid) and its derivatives, such as Gantrez® acids, Gantrez® salts (for example, sodium, calcium, magnesium, zinc), and Gantrez® anhydride. As used herein, the term “Gantrez® double or triple salts” refers to the copolymer of methylvinyl ether-co-maleic acid neutralized by two or more sodium, calcium, magnesium and/or zinc ions. In one embodiment, the composition comprises 0.5 to 10 wt. % Gantrez® salt polymer.

A fourth suitable polymer material is selected from polyacrylic acid or polymethacrylic acid homopolymers and copolymers and their derivatives, such as Carbopol® polymers.

A fifth suitable polymer material is selected from water-soluble or water dispersible natural polymers and their derivatives, such as sodium alginate, karaya gum, xanthan gum, locust bean gum, guar gum and its derivatives, pectin and its derivatives, chitosan and its derivatives, and carrageenan and its derivatives.

Further suitable polymer materials are selected from polyvinylpyrrolidone, copolymers of vinylpyrrolidone and their derivatives, such as Plasdone® and Polyplasdone® or polyvinylalcohol and its derivatives, such as Kollicoat® polymers, or combination thereof. In one embodiment, the composition comprises 5 to 20 wt. % Plasdone® polymer.

In one embodiment of this invention, the polymer is a polymer combination of HPMC and PEO. In a second embodiment of the invention, the polymer is a polymer combination of HPMC, PEO and a poly(methylvinyl ether-co-maleic acid) copolymer. In another embodiment of the invention, the polymer is a polymer combination of HPMC, PEO, polyvinylalcohol and polyvinylpyrrolidone.

The above components are used in safe and adhesively effective amounts, which herein means an amount sufficient to provide adherence to the oral cavity. In one embodiment of a denture adhesive cream formulation, the composition comprises from about 50 to 80 wt. % water, 8 to 20 wt. % HPMC and 5 to 20 wt. % polyethylene oxide polymer. In one embodiment of a denture adhesive liner formulation, the composition comprises 8 to 50 wt. % HPMC polymer and 5 to 30 wt. % polyethylene oxide polymer.

Suitably, in one embodiment of the invention, when the denture adhesive vehicle is glycerin alone, the ratio of HPMC to PEO is between 1:1 to 10:1 in glycerin system.

In addition to the ingredients indicated above, the instant composition may optionally contain other components to aid in enhancing the adhesive nature of the base components, including those commonly known and used in the adhesive art. Examples include, but are not limited to dicalcium phosphate, Gantrez acid and nanoclay/Montmorillonites.

In addition to the foregoing materials, the denture adhesive composition may be formulated with additional components well-known in the denture adhesive art including plasticizers, rheology modifiers, preservatives, humectants, emulsifiers, antioxidants, super-disintegrants or absorbents, for example, homopolymers of polyvinylpyrrolidone or copolymers of vinylpyrrolidone, flavoring agents, colorants, cross-linking agents, antimicrobial agents, control release agents, antifoaming agents, sweetening agents, viscosity modifiers and so forth.

Flavoring agents well known to the denture adhesive art may be added to the compositions of the present invention. These flavoring agents include without limitation, synthetic flavor oils and/or oils derived from plants, leaves, flowers, fruits and so forth, and combinations thereof. Representative flavor oils include, spearmint oil, cinnamon oil, oil of wintergreen (methylsalicylate) and peppermint oils. Also useful are artificial, natural or synthetic fruit flavors such as citrus oil including lemon, orange, grape, lime, and grapefruit, and fruit essences including apple, strawberry, cherry, pineapple, and so forth. The flavoring agent may be a liquid, spray dried, encapsulated, or absorbed on a carrier, and mixtures thereof. One embodiment of this invention contains as a flavoring agent, peppermint oil. The amount of flavoring agent utilized varies depending on such factors as flavor type, adhesive formulation and strength desired. In general, amounts of about 0.01 to about 5.0 wt. % of the total denture adhesive composition are suitable. In one embodiment of the invention, an amount of about 0.05 to 0.15 wt. % is used. In another embodiment, an amount of about 0.0 to about 0.1 wt. % is used.

Preservatives which may be used in the denture adhesive formulations of the invention include those known antimicrobial agents conventionally employed in the art, such as benzoic acid and sodium benzoate; the parabens; sorbic acid and sorbates; propionic acid and propionates; acetic acid and acetates; nitrates and nitrites; sulfur dioxide and sulfites; antibiotics; diethyl pyrocarbonate; epoxides; hydrogen peroxide; and phosphates. The parabens include the methyl, ethyl, propyl, and butyl esters of parahydroxybenzoic acid. Methyl paraben and propyl paraben are useful in one or more embodiment of the invention, utilized in amounts of about 0.03 to about 0.06 wt. % of the total denture adhesive composition.

The denture adhesive compositions may also include the use of sweeteners well known in the art. The sweetening agent may be selected from a wide range of materials including water-soluble agents, water-soluble artificial sweeteners, and dipeptide based sweeteners, including mixtures thereof. Representative sweeteners include without limitation, (a) water-soluble sweetening agents such as monosaccharides, disaccharides and polysaccharides such as xylose, ribose, glucose, mannose, galactose, fructose, dextrose, sucrose, sugar, maltose, partially hydrolyzed starch, or corn syrup solids and sugar alcohols such as sorbitol, xylitol, mannitol, maltitol, hydrogenated starch hydrolysate, and mixtures thereof; (b) water-soluble artificial sweeteners such as the soluble saccharin salts, i.e., sodium or calcium saccharin salts, cyclamate salts, acesulfam-K, sucralose, and the like, and the free acid form of saccharin; and (c) dipeptide based sweeteners such as L-aspartyl-L-phenylalanine methyl ester, and the like. In general, the amount of sweetener may be about 0.001 to about 5 wt. % of the total denture adhesive composition.

The colorants useful in the present invention include the pigments such as titanium dioxide, and may also include dyes suitable for food, drug and cosmetic applications. These colorants are known as FD&C dyes. Illustrative examples include without limitation, indigo dye, known as FD&C Blue No. 2, which is the disodium salt of 5,5′-indigotindi-sulfonic acid; FD&C Green No. 1, comprising a triphenylmethylene dye and is the monosodium salt of the 4-[4-N-ethyl-p-sulfobenzylamino) diphenylmethylene]-[1-(N-ethyl-N—P-sulfobenzyl)-2,5-cyclohexadienimine]. One embodiment of the invention uses FD&C Red No. 3 as a colorant.

The viscosity modifiers useful herein include without limitation, quaternary ammonium compounds and similar agents, starches, gums, casein, gelatin and semi-synthetic cellulose.

The composition of the present invention may also be used as a denture adhesive and/or bioadhesive and comprise one or more therapeutic actives suitable for mucosal or topical administration. The phrase “suitable for mucosal or topical administration,” as used herein, describes agents which are pharmacologically active when absorbed through internal mucosal surfaces of the body such as the oral cavity, or applied to the surfaces of the skin. Therapeutic actives may be present at a level from about 0 to about 40 wt. % of the composition.

Therapeutic actives that are useful in the instant compositions include antimicrobial agents such as iodine, sulfonamides, bisbiguanides, triclosan or phenolics; antibiotics such as tetracycline, neomycin, kanamycin, metronidazole, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, potassium chloride, strontium chloride or sodium fluoride; anesthetic agents such as lidocaine or benzocaine; antifungals; aromatics such as camphor, eucalyptus oil, and aldehyde derivatives such as benzaldehyde; insulin; steroids; and anti-neoplastics. It is recognized that in certain forms of therapy, combinations of these agents in the same delivery system may be useful in order to obtain an optimal effect. Thus, for example, an antimicrobial and an anti-inflammatory agent may be combined in a single delivery system to provide combined effectiveness.

The denture adhesive compositions may be in the form of a powder, a paste, a cream, a gel or a liner. These pastes or gels can either be applied by consumers from a container such as a tube, a brush pen, a spray bottle, a glue stick, or any other specially designed container with a consumer use friendly applicator, or can be fabricated into hydrogel films or hydrogel sheets, hydrogel strips or hydrogel wafers. These films or strips will possess a certain desirable thickness, strength and integrity during their application.

Means for preparing such formulations are well known in the denture adhesive art, employing conventional types of mixing equipment for blending, heating, and cooling solids and liquids. In one embodiment, the process of making a gel or paste formulation comprises the steps of: preparing a dry polymer powder mixture; preparing the medium such as water, glycerin or mixture of water/glycerin; adding the pre-made polymer powder mixture into the liquid medium and mixing until a uniform gel or paste is formed; and optionally at the end of mixing, a process such as vacuum to remove the air trapped in the product can be applied.

In the powder form, the components are admixed with flavoring agents and colorants, together with other ingredients such as non-toxic anti-caking agents (silica, magnesium stearate, talcum powder, and the like). The mixture of ingredients is thoroughly agitated or stirred to yield a generally homogenous intermixing of all components.

In the liner or layer form, the components are uniformly mixed and then coated onto a non-adhesive self supporting coating layer by any conventional coating techniques, such as by spraying (if the material is liquid or slurry or dissolved or suspended in a liquid such as water) or by sifting (if the denture adhesive is in powder form). In another embodiment, the components are admixed with the previously described preservatives, flavoring agents, colorants, sweetening agents, viscosity modifiers, and so forth. The liner is then formed by any variety of techniques known in the polymer film-forming art, including casting, calendaring, coating, and extrusion. In one embodiment to form liners, the components are first mechanically softened by a ring roller; smoothed on a hydraulic press, and die-cut as desired into denture liner shapes or other desired shapes.

### EXAMPLES

To further illustrate the invention, Examples are set forth below. In these, as throughout the specification and claims, all parts and percentages are by weight and all temperatures in degrees centigrade, unless otherwise indicated.

### Example 1

2134.4 grams of distilled water were heated to 90° C. in a glass stirred reaction vessel with water jacket. Then the pre-made powder mixture of 427 grams of hydroxypropyl methylcellulose (under the trade name of Benecel® from Aqualon Inc, Grade MP874), 214 grams of polyethylene oxide (under the trade name of PolyOx® from Dow Inc., Grade NF303), 171 grams of sodium/calcium partial mixed salt of methyl vinyl ether/maleic anhydride copolymer (“Gantrez® double salt”), 43 grams of methyl vinyl ether/maleic acid copolymer (“Gantrez® acid”), 2.8 grams of potassium sorbate and 2.8 grams of sodium benzoate were added slowly into the water and mixing continued until a uniformed gel paste was obtained. The reaction was then cooled to 40° C. before applying the vacuum to remove the air trapped in the product. Finally the product was cooled to room temperature and placed in a clean sterilized glass jar. (See table below)

| Ingredient | Wt (gm) | Formula Wt (%) |
|---|---|---|
| HPMC MP874 | 427 | 14.3 |
| PolyOx NF303 | 214 | 7.1 |
| Gantrez ® Double Salt | 171 | 5.7 |
| Gantrez ® Acid | 43 | 1.4 |
| Potassium Sorbate | 2.8 | 0.1 |
| Sodium Benzoate | 2.8 | 0.1 |
| H₂O | 2134.4 | 71.3 |
| Total | 2995 | 100.0 |

### Example 2

150 grams of distilled water and 250 grams of glycerin were mixed in a stainless bowl using a KitchenAid® mixer. Then the pre-made powder mixture of 100 grams of hydroxypropyl methylcellulose (under the trade name of Benecel® from Aqualon Inc, Grade MP874), 50 grams of polyethylene oxide (under the trade name of PolyOx® from Dow Inc., Grade NF303), 100 grams of vinyl acetate/vinyl pyrrolidone copolymer (under the trade name of Plasdone® S630 from ISP Inc.), 0.6 grams of potassium sorbate and 0.6 grams of sodium benzoate was added slowly into the water/glycerin mixture and mixing continued until a uniformed paste was obtained. A vacuum was then applied to remove the air trapped in the product. Finally the product was placed in a clean sterilized glass jar. (See table below)

| Ingredients | Wt (gm) | Formula Wt (%) |
|---|---|---|
| HPMC MP874 | 100 | 15.4 |
| PolyOx NF303 | 50 | 7.7 |
| PLASDONE 630 | 100 | 15.4 |
| Potassium Sorbate | 0.6 | 0.1 |
| Sodium Benzoate | 0.6 | 0.1 |
| Glycerin | 250 | 38.4 |
| H₂O | 150 | 23.0 |
| Total | 651.2 | 100 |

### Example 3

229.8 grams of distilled water were added into a stainless bowl using a KitchenAid® mixer. Then the pre-made powder mixture of 66 grams of hydroxypropyl methylcellulose (under the trade name of Benecel® from Aqualon Inc, Grade MP874), 33 grams of polyethylene oxide (under the trade name of PolyOx® from Dow Inc., Grade NF303) and 0.2 grams of sodium benzoate were added slowly into the water and mixing continued until a uniformed paste was obtained. A vacuum was then applied to remove the air trapped in the product. Finally the product was placed in a clean sterilized glass jar. (See table below)

| Ingredient | Wt (gm) | Formula Wt (%) |
|---|---|---|
| HPMC MP874 | 66 | 20.1 |
| PolyOx NF303 | 33 | 10.0 |
| Na Benzoate | 0.2 | 0.1 |
| H₂O | 229.8 | 69.8 |
| Total | 329 | 100 |

### Example 4

150 grams of distilled water and 250 grams of glycerin were mixed in a stainless bowl using a KitchenAid® mixer. 0.6 grams of Peppermint Oil and 0.6 grams of Spearmint Oil were added into the above mixture and mixed. Then the pre-made powder mixture of 100 grams of hydroxypropyl methylcellulose (under the trade name of Benecel® from Aqualon Inc, Grade MP874), 50 grams of polyethylene oxide (under the trade name of PolyOx® from Dow Inc., Grade NF303), 100 grams of vinyl acetate/vinyl pyrrolidone copolymer (under the trade name of Plasdone® S630 from ISP Inc.), were added slowly into the water/glycerin mixture and mixing continued until a uniformed paste was obtained. A vacuum was then applied to remove the air trapped in the product. Finally the product was placed in a clean sterilized glass jar. (See table below)

| Ingredients | Wt (gm) | Formula Wt (%) |
|---|---|---|
| PLASDONE 630 | 100 | 15.4 |
| PolyOx NF303 | 50 | 7.7 |
| HPMC MP874 | 100 | 15.4 |
| H₂O | 150 | 23.0 |
| Peppermint Oil | 0.6 | 0.1 |
| Spearmint Oil | 0.6 | 0.1 |
| Glycerin | 250 | 38.4 |
| Total | 651.2 | 100 |

### Example 5

155.2 grams of glycerin were added into a stainless bowl using a KitchenAid® mixer. 5.2 grams of fumed silica was added into the above mixture and mixed. Then the pre-made powder mixture of 40 grams of hydroxypropyl methylcellulose (under the trade name of Benecel® from Aqualon Inc, Grade MP874), 20 grams of polyethylene oxide (under the trade name of PolyOx® from Dow Inc., Grade NF303), 20 grams of vinyl acetate/vinyl pyrrolidone copolymer (under the trade name of Plasdone® S630 from ISP Inc.), 20 grams of sodium/calcium partial mixed salt of methyl vinyl ether/maleic anhydride copolymer (“Gantrez® double salt”), 0.24 grams of potassium sorbate, 0.24 grams of sodium benzoate and 0.04 grams of benzoic acid were added slowly into the glycerin/fumed silica mixture and mixing continued until a uniformed paste was obtained. A vacuum was then applied to remove the air trapped in the product. Finally the product was placed in a clean sterilized glass jar. (See table below)

| Ingredients | Wt (gm) | Formula Wt (%) |
|---|---|---|
| PolyOx NF303 | 20 | 7.67 |
| HPMC MP874 | 40 | 15.33 |
| PLASDONE 630 | 20 | 7.67 |
| Gantrez ® Double Salt | 20 | 7.67 |
| Fumed Silica | 5.2 | 1.99 |
| K Sorbate | 0.24 | 0.09 |
| Na Benzoate | 0.24 | 0.09 |
| Benzoic Acid | 0.04 | 0.02 |
| Glycerin | 155.2 | 59.48 |
| Total | 260.92 | 100.00 |

### Example 6

125 grams of glycerin were added into a stainless bowl using a KitchenAid® mixer. Then the pre-made powder mixture of 50 grams of hydroxypropyl methylcellulose (under the trade name of Benecel® from Aqualon Inc, Grade MP874), 25 grams of polyethylene oxide (under the trade name of PolyOx® from Dow Inc., Grade NF303), 50 grams of vinyl acetate/vinyl pyrrolidone copolymer (under the trade name of Plasdone® S630 from ISP Inc.), 2.5 grams of Montmorillonites (under the trade name of NanoClay® PGV from Nanocor Inc.), 0.24 grams of potassium sorbate, 0.24 grams of sodium benzoate and 0.04 grams of benzoic acid were added slowly into the glycerin and mixing continued until a uniformed paste was obtained. A vacuum was then applied to remove the air trapped in the product. Finally the product was placed in a clean sterilized glass jar. (See table below)

| Ingredients | Wt (gm) | Formula Wt (%) |
|---|---|---|
| HPMC MP874 | 50 | 19.76 |
| PolyOx NF303 | 25 | 9.88 |
| PLASDONE 630 | 50 | 19.76 |
| K Sorbate | 0.24 | 0.09 |
| Na Benzoate | 0.24 | 0.09 |
| Benzoic Acid | 0.04 | 0.02 |
| Nanoclay-PGV | 2.5 | 0.99 |
| Glycerin | 125 | 49.40 |
| Total | 253.02 | 100 |

### Example 7

298.7 grams of propylene glycol were added into a stainless bowl using a KitchenAid® mixer. Then the pre-made powder mixture of 74.55 grams of hydroxypropyl methylcellulose (under the trade name of Benecel® from Aqualon Inc, Grade MP874), 59.65 grams of polyethylene oxide (under the trade name of PolyOx® from Dow Inc., Grade NF303), 67.1 grams of vinyl acetate/vinyl pyrrolidone copolymer (under the trade name of Plasdone® S630 from ISP Inc.), 15 grams of Montmorillonites (under the trade name of NanoClay® PGV from Nanocor Inc.), 0.24 grams of potassium sorbate, 0.24 grams of sodium benzoate and 0.04 grams of benzoic acid were added slowly into the propylene glycol and mixing continued until a uniformed paste was obtained. A vacuum was then applied to remove the air trapped in the product. Finally the product was placed in a clean sterilized glass jar. (See table below)

| Ingredients | Wt (gm) | Formula Wt (%) |
|---|---|---|
| HPMC MP874 | 74.55 | 14.46 |
| PolyOx NF303 | 59.65 | 11.57 |
| PLASDONE 630 | 67.1 | 13.02 |
| K Sorbate | 0.24 | 0.05 |
| Na Benzoate | 0.24 | 0.05 |
| Benzoic Acid | 0.04 | 0.01 |
| Nanoclay-PGV | 15 | 2.91 |
| Propylene Glycol | 298.7 | 57.94 |
| Total | 515.52 | 100 |

### Examples 8 Through 12

Similarly to Examples 1 through 7, the following formulations were made and are included within the scope of this invention.

| | Formula 8 | Formula 9 | Formula 10 | Formula 11 | Formula 12 |
|---|---|---|---|---|---|
| Ingredients | Wt (%) | Wt (%) | Wt (%) | Wt (%) | Wt (%) |
| HPMC MP874 | 15.1 | 15.4 | 15.4 | 21.94 | 15.4 |
| PolyOx NF303 | 7.6 | 7.7 | 9.7 | 3.48 | 7.7 |
| PLASDONE 630 | | 15.4 | 15.4 | 12.2 | 15.4 |
| Gantrez Salt (Na/Ca) | 6.1 | | | | 6.3 |
| Gantrez Acid S97 BF | 1.6 | | | | |
| K Sorbate | | 0.09 | 0.09 | 0.08 | |
| Na Benzoate | | 0.09 | 0.09 | 0.08 | |
| Benzoic Acid | | 0.02 | 0.02 | | |
| Nanoclay-PGV | | | | 5.65 | |
| Propylene Glycol | | | 57.49 | | |
| Glycerin 99.7%, USP | | 38.3 | | 56.56 | 55.2 |
| Aerosil 200 (SiO₂) | | | 1.0 | | |
| Sodium Saccharin USP | | | 0.01 | 0.01 | |
| Flavor | | | 0.08 | | |
| Purified Water | 69.6 | 23 | | | |
| Total | 100.0 | 100.0 | 100.0 | | |

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A denture adhesive composition free of a hydrocarbon vehicle including mineral oil or petrolatum.

2. The denture adhesive composition as claimed in claim 1, wherein the vehicle is selected from water, glycerin, propylene glycol, a low molecular weight polyethylene glycol, alone and in combinations thereof.

3. The denture adhesive composition as claimed in claim 1, having a combination of at least two polymers, one which has adhesive properties, the other which has cohesive properties.

4. The denture adhesive composition as claimed in claim 3, wherein the combination of polymers is polyethyleneoxide and hydroxypropylmethyl cellulose.

5. The denture adhesive composition as claimed in 2, wherein the vehicle is glycerin.

6. The denture adhesive composition as claimed in claim 5, wherein the combination of polymers is polyethyleneoxide and hydroxypropylmethyl cellulose.

7. The denture adhesive composition as claimed in claim 6, wherein the polymers are found in a ratio between 1:1 and 10:1 of hydroxypropylmethylcellulose to polyoxyethylene.

8. The denture adhesive composition as claimed in claim 2, wherein the vehicle is water.

9. The denture adhesive composition as claimed in claim 2, wherein the vehicle is a combination of water and glycerin.

10. The denture adhesive composition as claimed in claim 2, wherein the vehicle is propylene glycol.
